(19) United States
(12) Patent Application Publication
(10) Pub. No.: US 2022/0304611 A1
(43) Pub. Date: Sep. 29, 2022

(54) ATRIAL FIBRILLATION DETECTION DEVICE, METHOD, SYSTEM AND STORAGE MEDIUM

(71) Applicants: Shanghai First People's Hospital, Shanghai (CN); Shanghai Jiao Tong University, Shanghai (CN)

(72) Inventors: Liqun ZHAO, Shanghai (CN); Chengliang LIU, Shanghai (CN); Jinlei LIU, Shanghai (CN); Fei ZHANG, Shanghai (CN)

(21) Appl. No.: 17/840,447

(22) Filed: Jun. 14, 2022

Related U.S. Application Data
(63) Continuation of application No. PCT/CN2020/120138, filed on Oct. 10, 2020.

(51) Int. Cl. A61B 5/361; A61B 5/352; A61B 5/36; A61B 5/366

(52) U.S. Cl. CPC A61B 5/361; A61B 5/352; A61B 5/36; A61B 5/366

(57) ABSTRACT

The present invention discloses an atrial fibrillation detection device, method, system and storage medium. The device includes an ECG signal processing module, configured to identify positions of Q, R, S and T points of all heartbeats in an ECG signal acquired in a preset time, and determine a RR interval, a P point amplitude, an R point amplitude and a TQ segment waveform of each heartbeat according to the positions of the P, Q, R, S and T positions; and a detection module, configured to acquire an integrated score and perform conditional judgment on the integrated score, wherein the fifth score is a quotient of a total number of f waves in all the TQ segment waveforms in the ECG signal to a total number of the TQ segment waveforms involved in the ECG signal.

### CROSS-REFERENCE TO RELATED APPLICATIONS

This application is a continuation application of International Patent Application No. PCT/CN2020/120138, filed on Oct. 10, 2020, and entitled “ATRIAL FIBRILLATION DETECTION DEVICE, METHOD, SYSTEM AND STORAGE MEDIUM”. The above-referenced applications are incorporated herein by reference in their entirety.

### TECHNICAL FIELD

The present invention relates to the field of electrocardiogram (ECG) monitoring, in particular to anatrial fibrillation detection device, method, system and storage medium.

### BACKGROUND

Atrial fibrillation is a common arrhythmia with an incidence of higher than 10% in people over 80 years old. The atrial fibrillation is an atrial arrhythmia induced by many small reentrant loops caused by an atrial dominant reentrant loop; when it occurs, regular and orderly atrial activities loses and are replaced by rapid and disordered fibrillation waves. Patients often feel flustered and fatigued due to irregular heartbeats. Moreover, atrial fibrillation can be found in all patients with organic heart disease, with a high incidence and long duration; it may also worsen cardiac functions and cause serious cardiovascular complications, such as heart failure and arterial embolism, resulting in disability of patients or an increased death rate. Therefore, effective detection of atrial fibrillation at an earlier stage is beneficial for treatment and health monitoring.

Usually, ECG is used for observing changes in cardiac potential and diagnosing cardiovascular diseases. Atrial fibrillation can also be diagnosed by ECG. However, since the changes in the amplitude and frequency components of the ECG waveform are tiny, it is difficult and time-consuming for doctors to diagnose cardiovascular diseases by ECG. In addition, some patients with atrial fibrillation have paroxysmal atrial fibrillation, which may not always attack during detection in a hospital.

Currently, patients with atrial fibrillation are mostly diagnosed by a doctor through detection in a hospital ECG room. For paroxysmal atrial fibrillation that is hard to capture, a 24-hour dynamic electrocardiograph is adopted to collect ECG continuously, and the data transmitted to the hospital for diagnosis by a doctor after 2-3 days. Therefore, it is difficult to detect patients with atrial fibrillation, and the detection effect is not accurate enough.

### SUMMARY

The present invention is intended to provide an atrial fibrillation detection device, method, system and storage medium, through which an integrated score is obtained through four models, and conditional judgment is performed according to the integrated score and a fifth score to obtain a suspected degree of suffering from atrial fibrillation. The atrial fibrillation detection device provided by the present invention can efficiently and accurately determine whether a patient suffers from atrial fibrillation, and can obtain a severity of the disease; it is more convenient for judging patients' state of illness, so that patients can receive timely treatment when they just suffer from mild atrial fibrillation.

In order to solve the above-mentioned problems, in the first aspect, the present invention provides an atrial fibrillation detection device. The device includes: an ECG signal processing module, configured for identifying positions of P, Q, R, S and T points of all heartbeats in an ECG signal acquired in a preset time and determining a RR interval, a P point amplitude, an R point amplitude and a TQ segment waveform of each heartbeat according to the positions of the P, Q, R, S and T points; and

a detection module, configured for performing conditional judgment on an extremal ratio of RR intervals in an ECG signal through a first model to obtain a first score, performing conditional judgment on a ratio of a number of the RR intervals with a deviation value exceeding a standard deviation to a number of all the RR intervals in the ECG signal through a second model to obtain a second score, performing conditional judgment on a number of the RR interval groups similar to other arrhythmia in the ECG signal through a third model to obtain a third score, performing conditional judgment on a ratio of a number of heartbeat waveforms with a normal PR height ratio to a number of all heartbeat waveforms in the ECG signal through a fourth model to obtain a fourth score, integrating the first score, the second score, the third score and the fourth score to obtain an integrated score, and performing conditional judgment on the integrated score and a fifth score to determine a suspected degree of suffering from atrial fibrillation, wherein and the fifth score is a quotient of a total number of waveforms greater than an amplitude threshold of respective f waveform and with a width greater than a width threshold in all the TQ segment waveforms in the ECG signal to a total number of the TQ segment waveforms involved in the ECG signal.

In the second aspect, the present invention provides an atrial fibrillation detection method, including the following steps: identifying positions of P, Q, R, S and T points of all heartbeats in an ECG signal acquired in a preset time and determining a RR interval, a P point amplitude, an R point amplitude and a TQ segment waveform of each heartbeat according to the positions of the P, Q, R, S and T points; performing conditional judgment on an extremal ratio of RR intervals in an ECG signal through a first model to obtain a first score; performing conditional judgment on a ratio of a number of the RR intervals with a deviation value exceeding a standard deviation to a number of all the RR intervals in the ECG signal through a second model to obtain a second score; performing conditional judgment on a number of the RR interval groups similar to other arrhythmia in the ECG signal through a third model to obtain a third score; performing conditional judgment on a ratio of a number of heartbeat waveforms with a normal PR height ratio to a number of normal heartbeat waveforms in the ECG signal through a fourth model to obtain a fourth score; integrating the first score, the second score, the third score and the fourth score to obtain an integrated score; performing conditional judgment on the integrated score and a fifth score to determine a suspected degree of suffering from atrial fibrillation, wherein the fifth score is a quotient of a total number of f waves in all the TQ segment waveforms in the ECG signal to a total number of the TQ segment waveforms involved in the ECG signal; wherein the f waves in each of the TQ segment waveforms are the waveforms greater than an amplitude threshold of respective f waveform and with a width greater than a width threshold.

### BRIEF DESCRIPTION OF DRAWINGS

FIG. 1 is an ECG signal pattern schematically provided by an embodiment of the present invention;

FIG. 2 is a structural diagram of an atrial fibrillation detection device provided by an embodiment of the present invention;

FIG. 3 is a flow chart for acquiring a first score by a first model provided by an embodiment of the present invention;

FIG. 4 is a flow chart for acquiring a second score by a second model provided by an embodiment of the present invention;

FIG. 5 is a flow chart for acquiring a third score by a third model provided by an embodiment of the present invention;

FIG. 6 is a flow chart for acquiring a fourth score by a fourth model provided by an embodiment of the present invention;

FIG. 7 is a flow chart for acquiring a fifth score by a fifth model provided by an embodiment of the present invention;

FIG. 8 is a flow chart of an atrial fibrillation detection method provided by an embodiment of the present invention.

### DESCRIPTION OF EMBODIMENTS

To make the objectives, technical solutions and advantages of the present invention more clearly, the present invention will be further explained in detail below in combination with the specific embodiments and the accompanying drawings. It shall be understood that these descriptions are exemplary only, rather than limiting the scope of the present invention. In addition, in the explanations below, descriptions of well-known structures and techniques are omitted, in order to avoid unnecessarily mistaking concepts of the present invention.

It is obvious that the described embodiments are parts of, rather than all of, the embodiments of the present invention. On the basis of the embodiments in the present invention, all the other embodiments obtained by those of ordinary skill in the art without creative efforts will fall within the protection scope of the present invention.

In the descriptions of the present invention, it shall be noted that, the terms of “first”, “second” and “third” are used for description only, but cannot be understood to indicate or imply relative importance.

In addition, the technical features described below and involved in different embodiments of the present invention can be combined with each other, as long as there is no conflict.

Before discussing the schemes of the present invention, relevant contents in the field firstly explained in details.

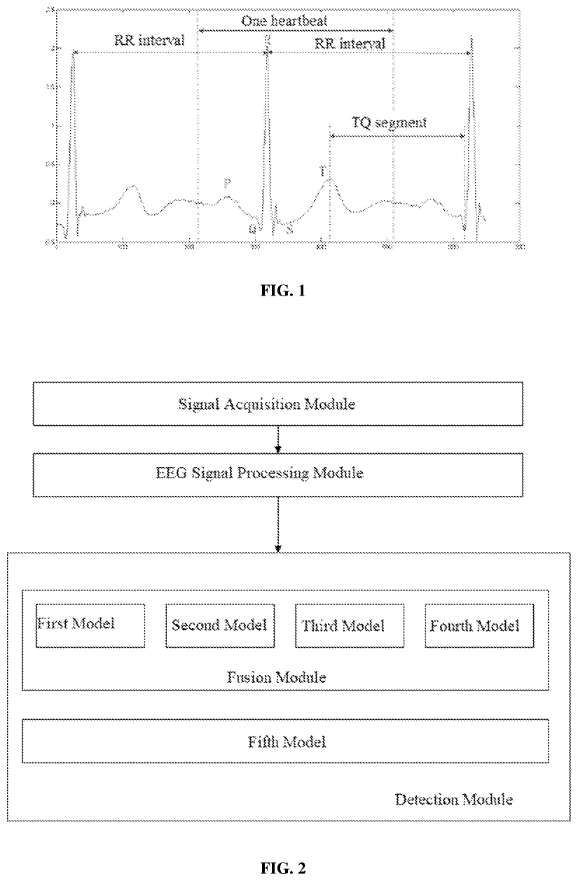

FIG. 1 is an ECG provided by an embodiment of the present invention.

As shown in FIG. 1, in the field, a segment of waveform before and after an R point is a heartbeat, a time interval between adjacent R points is called as an RR interval, a Q point is a wave trough before the R point, and a T point is a wave peak after the R point. The TQ segment in the present invention is from a T point to a Q point that is located after the T point and is the closest to the T point.

First of all, the medical basis for the diagnosis of atrial fibrillation is given, and the following two conditions must be satisfied at the same time.

(1) The ventricular rate is absolutely irregular, that is, the RR interval is absolutely irregular, in other words, the values of many consecutive RR intervals are not equal without regular changes.

(2) P waves disappear and are replaced by quite irregular flutter waves (f waves) in different sizes and shapes and at different intervals. That is to say, P waves disappear and f waves appear.

Therefore, in the prior art, detection devices usually only consider the different values of RR intervals within a certain range to reflect the irregular ventricular rate, but it is difficult to reflect the disappearance of the p waves and the appearance of the f waves by an algorithm, and thus there is less detection for disappearance of the p waves and the appearance of the f waves in the prior art. Accordingly, it is inaccurate in the prior art to determine whether a patient suffers from atrial fibrillation.

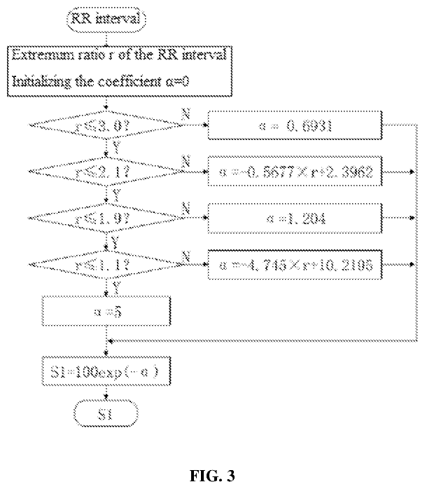

FIG. 2 is a structural diagram of an atrial fibrillation detection device provided by an embodiment of the present invention.

As shown in FIG. 2, the atrial fibrillation detection device includes an ECG signal processing module and a detection module.

Wherein the ECG signal processing module is configured for identifying positions of P, Q, R, S and T points of all heartbeats in an ECG signal acquired in a preset time and determining a RR interval, a P point amplitude, an R point amplitude and a TQ segment waveform of each heartbeat according to the positions of the P, Q, R, S and T points of each heartbeat.

It can be understood that the ECG signal acquired within a preset time herein can refer to an ECG signal collected historically or an ECG signal collected in real time; or the ECG signal herein can either be collected by a signal acquisition module in the atrial fibrillation detection device, or collected by other external devices and then inputted to the ECG signal processing module of the atrial fibrillation detection device.

Wherein the preset time is preferably 20 s.

Wherein the detection module is configured for performing conditional judgment on an extremal ratio of RR intervals in an ECG signal through a first model to obtain a first score, performing conditional judgment on a number of the RR intervals with a deviation value exceeding a standard deviation and a ratio of all the RR intervals in the ECG signal through a second model to obtain a second score, performing conditional judgment on a number of the RR interval groups similar to other arrhythmia in the ECG signal through a third model to obtain a third score, performing conditional judgment on a ratio of a number of heartbeat waveforms with a normal PR height ratio to a number of normal heartbeat waveforms in the ECG signal through a fourth model to obtain a fourth score, integrating the first score, the second score, the third score and the fourth score to obtain an integrated score, and performing conditional judgment on the integrated score and a fifth score to determine a suspected degree of suffering from atrial fibrillation, wherein the fifth score is a quotient of a total number of waveforms greater than an amplitude threshold of respective f waveform and with a width greater than a width threshold in all the TQ segment waveforms in the ECG signal to a total number of the TQ segment waveforms involved in the ECG signal, and wherein the f waves in each of the TQ segment waveforms are the waveforms greater than an amplitude threshold of respective f waveform and with a width greater than a width threshold.

In an embodiment, a signal acquisition module is arranged in the atrial fibrillation detection device of the present invention.

The signal acquisition module is connected with the ECG signal processing module.

Wherein the signal acquisition module is configured for collecting ECG signals every preset time; and the ECG signal includes a lead-II ECG signal and a lead-V1 ECG signal.

Wherein the signal acquisition module collects ECG signals every preset time. For example, portable hardware collected an original ECG signal from a human body surface and processed the original ECG signal to remove interference to obtain an available EGC signal. It can be set that the signal acquisition module collects ECG signals every 20 s as a subject.

Specifically, the signal acquisition module processes the original ECG, including filtering with a wavelet threshold method to eliminate noise. Specifically, an acquired ECG signal is decomposed into 8 layers using a db6 wavelet. A wavelet coefficient obtained by decomposition is processed by a soft threshold method to obtain a modified wavelet coefficient. The signal is then reconstructed by the modified wavelet coefficient to obtain a usable ECG signal.

In an embodiment, the ECG signal processing module is configured for identifying the positions of P, Q, R, S and T points of each heartbeat in an obtained ECG signal, including:

The ECG signal processing module detects the main feature points of the ECG signal based on a biorthogonal wavelet and a first-order difference. The main steps include:

detecting a QRS complex in a lead-II ECG signal using a B-spline biorthogonal wavelet to determine the positions of the Q, R and S points;

identifying the lead-II ECG signal using the first-order difference to obtain the positions of the P and T points;

obtaining waveforms of all TQ segments based on the positions of the T point and the nearest Q point after the T point and the lead-V1 ECG;

obtaining RR intervals between heartbeats from the positions of all R points and the lead-II ECG signal;

obtaining P point amplitudes of the heartbeats from the positions of all P points and the lead-II ECG signal; and

obtaining R point amplitudes of the heartbeats from the positions of all R points and the lead-II ECG signal.

In a preferable embodiment, the ECG signal processing module is also configured for removing a RR interval that is greater than 0.5 times the mean value and less than 1.6 times the mean value within a preset time (20 s).

Specifically, a mean value of all RR intervals is calculated. Then each RR interval is judged whether it is greater than 0.5 times the mean value and less than 1.6 times the mean value. If the condition is not satisfied, the RR interval is deemed as an abnormal value and eliminated.

Specifically, the detection module includes a first model, a second model, a third model, a fourth model, a fusion module and a fifth model.

FIG. 2 is a flow chart for acquiring a first score by a first model provided by an embodiment of the present invention.

As shown in FIG. 2, for the first model, it is necessary to input a maximum-to-minimum ratio (extremal ratio) of each RR interval, determine the maximum values and the minimum values of all RR intervals, determine the maximum-to-minimum ratios of the RR intervals, determine a range of the maximum-to-minimum ratios of the RR intervals, responds to the range of the maximum-to-minimum ratios of the RR intervals, and calculate a coefficient to calculate a score of the first model base on the coefficient.

Specifically, the first model is S1=100 exp(−α), where S1 is the first score, and α is a coefficient of the first model.

For the detection module, the step of performing conditional judgment on an extremal ratio of RR intervals in an ECG signal through a first model to obtain a first score includes: a ratio r of a length of a maximum RR interval to that of a minimum RR interval is obtained according to all RR intervals in an inputted ECG signal.

It is judged whether r is less than or equal to 3.0. When the ratio r is greater than 3, the coefficient is determined as 0.6931.

Then, it is judged whether the ratio r is less than or equal to 2.1. when the ratio r is less than or equal to 3 and greater than 2.1, α is determined as −0.5677×r+2.3962.

Then, it is judged whether the ratio r is less than or equal to 1.9. When the ratio r is less than or equal to 2.1 and greater than 1.9, α is determined as 1.204.

Then, it is judged whether the ratio r is less than or equal to 1.1. When the ratio r is less than or equal to 1.9 and greater than 1.1, α is determined as −4.745×r+10.2195.

When the ratio r is less than or equal to 1.1, α is determined as 5.

It shall be noted that in the first model, it is considered that the difference between the maximum value and the minimum value of RR intervals can reflect a degree of change in the RR intervals and further reflect a degree of evenness of the RR intervals. Considerations is also given to the differences in the RR interval values of different individuals, the maximum-to-minimum ratio is used as an indicator r of the first model. The greater the r value is, the greater the degree of unevenness of the RR intervals is.

By adopting the multiple judgment criteria for the r value, it can better reflect the difference between the maximum value and the minimum value of RR intervals, which can be reflected by the first score S1. The interval division effect is better than judging by simply setting a threshold. In addition to the middle interval, the existence can be definitely judged after it exceeds or is lower than a certain value, namely the two intervals corresponding to the beginning and the end.

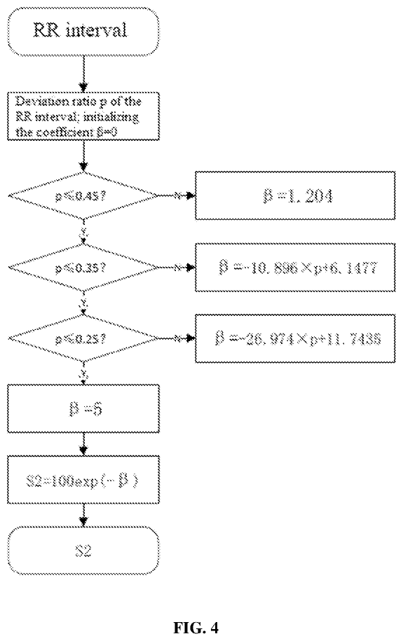

FIG. 3 is a flow chart for acquiring a second score by a second model provided by an embodiment of the present invention.

As shown in FIG. 3, for the second model, firstly it is necessary to obtain a mean value and a standard deviation of RR intervals according to all the RR intervals in an inputted ECG signal, and then calculate a deviation of each RR interval from the mean value. A ratio P of a number of the RR intervals with a deviation value exceeding the standard deviation to a number of all the RR intervals is determined. Then, a coefficient value of the second model is determined according to a numerical range of the ratio P, and a score S2 of the second model is calculated from the coefficient.

Specifically, the second model is S2=100 exp(−β), where S2 is the second score, and β is the coefficient of the second model.

Wherein the step of performing conditional judgment on a ratio p of a number of RR intervals with a deviation value exceeding a standard deviation to a number of all the RR intervals in the ECG signal through a second model to obtain a second score includes:

the deviation of each RR interval from the mean value is obtained according to all the RR intervals in an inputted ECG signal acquired in a preset time; and

the ratio p of the number of the RR intervals with the deviation exceeding the standard deviation to the number of all the RR intervals is determined.

First, it is judged whether the ratio p is less than or equal to 0.45, and when the ratio p is greater than 0.45, β is determined as 1.204.

Whether the ratio p is less than or equal to 0.45, it is judged whether the ratio p is less than or equal to 0.35.

When the ratio p is less than or equal to 0.45 and greater than 0.35, β is determined as −10.896×p+6.1477.

When the ratio p is less than or equal to 0.35, it is judged whether the ratio p is less than or equal to 0.25.

When the ratio p is less than or equal to 0.35 and greater than 0.25, β is determined as −26.974×p+11.7435.

When the ratio p is less than or equal to 0.25, β is determined as 5.

It shall be noted that, in the above second model, it is considered that if there are more RR intervals with greater changes, it can also indicate a greater degree of unevenness of the RR intervals. The present invention firstly calculates the mean value and the standard deviation of all the RR intervals, then calculates the deviation of each RR interval from the mean value, and adopts the ratio p of the number of the RR intervals with the deviation value exceeding the standard deviation to the number of all the RR intervals as an indicator for absolute unevenness. The greater the p value is, the more the RR intervals with greater changes as indicated, and the greater the degree of RR interval irregularity as further indicated. Setting multiple interval judgment can better indicate the number of the RR intervals with greater changes, making the detection effect more accurate.

FIG. 4 is a flow chart for acquiring a third score by a third model provided by an embodiment of the present invention.

Wherein for the third model, it is necessary to input a number of RR interval groups conforming to complete compensatory pause and approximate to the type of premature beats, process 4 consecutive RR intervals as one RR interval group to determine whether each RR interval group conforms to complete compensatory pause, determine whether each RR interval group is approximate to the type of premature beats, determine a number of RR interval groups conforming to complete compensatory pause and approximate to the type of premature beats, determine a range of the number of RR interval groups conforming to complete compensatory pause and approximate to the type of premature beats, respond to the range of the number of RR interval groups conforming to complete compensatory pause and approximate to the type of premature beats, calculate a coefficient value, and calculate a score of the third model from the coefficient.

Specifically, as shown in FIG. 4, the third model is S3=100 exp(−γ), where S3 is the third score, and γ is the coefficient of the third model.

Wherein for the detection module, the step of performing conditional judgment on a number of the RR interval groups similar to other arrhythmia in the ECG signal through a third model to obtain a third score includes:

four continuous RR intervals are taken as a RR interval group according to a time sequence, a sum of a second RR interval and a third RR interval is compared with a mean value of RR intervals in each RR interval group; and

if the sum of the second RR interval and the third RR interval is less than 2.2 times the mean value of RR intervals and greater than 1.1 times the mean value of RR intervals, it meet the judgment condition 1. Next, the four RR intervals are compared to determine whether they meet the judgment condition 2 for being similar to other arrhythmia If the first RR interval is greater than the second RR interval, the third RR interval is greater than the second RR interval, and the third RR interval is also greater than the fourth RR interval, it is determined to meet the judgment condition 2. The RR interval groups meeting both judgment conditions at the same time are those similar to other arrhythmia, and a number of the RR interval groups is recorded as n.

That is, if the sum of the second RR interval and the third RR interval is less than 2.2 times the mean value of RR intervals and greater than 1.1 times the mean value of RR intervals, the first RR interval is greater than the second RR interval, and the third RR interval is greater than the second RR interval and the fourth RR interval, the RR interval group is determined as a RR interval group similar to other arrhythmia

The coefficient γ is initialized as 0, then the value of number n is judged.

It is firstly judged whether n is less than or equal to 4. When the number n of RR interval groups similar to other arrhythmia is greater than 4, γ is determined to be 0.6931.

When the number n is less than or equal to 4, it is judged whether n is less than or equal to 3.

When the number n is less than or equal to 4 and greater than 3, γ is determined as 1.204.

When the number n is less than or equal to 3, it is judged whether n is less than or equal to 2.

When the number n is less than or equal to 3 and greater than 2, γ is determined as 1.8971.

When the number n is less than or equal to 2 and greater than 1, γ is determined as 2.9957.

When the number n is less than or equal to 1, γ is determined as 5.

It shall be noted that, if a RR interval manifests other regularities of arrhythmia, it means that although the RR interval is uneven, it is regular and not absolutely uneven, and thus it is not atrial fibrillation. In the third model, it is a judgment condition that judges whether the regularity of premature beats and escape beats (two heart diseases) is met. A number of the RR interval groups meeting the judgment condition is used as an indicator n of the third model. The greater the n value is, the more the RR intervals suspected of other arrhythmia as indicated, and the smaller the degree of absolute unevenness of RR intervals as further indicated.

It shall be noted that, the RR interval is an indicator of time, thus the value is the same for all the leads, and it can be calculated by II lead.

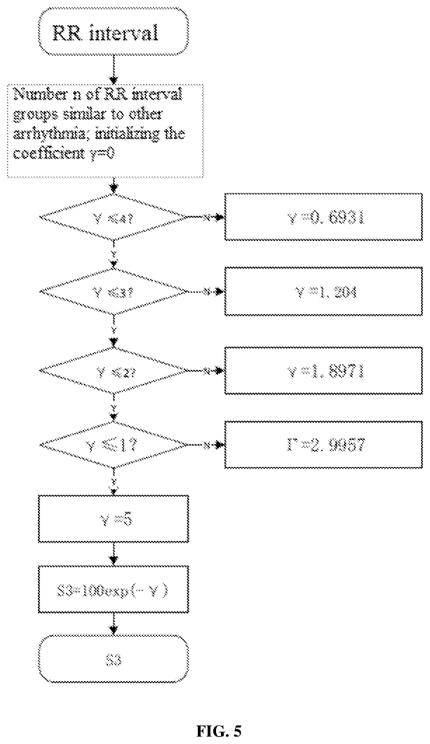

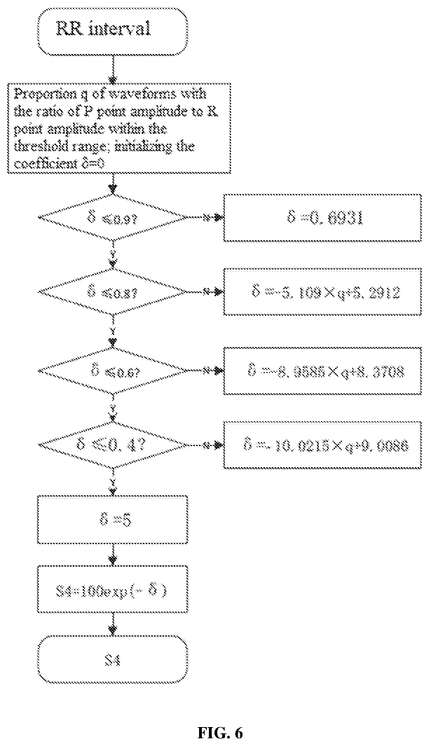

FIG. 5 is a flow chart for acquiring a fourth score by a fourth model provided by an embodiment of the present invention.

For the fourth model, it needs to input P point amplitudes and R point amplitudes of all waveforms within 20 s. Then, it needs to determine the PR height ratios of all waveforms, determine whether the PR height ratio of each waveform is within a threshold range, determine a proportion of the waveforms with the PR height ratio within the threshold range, determine a range of the proportion of the waveforms with the PR height ratio within the threshold range, respond to the range of the proportion, calculate a coefficient value, and calculate a score of the fourth model from the coefficient.

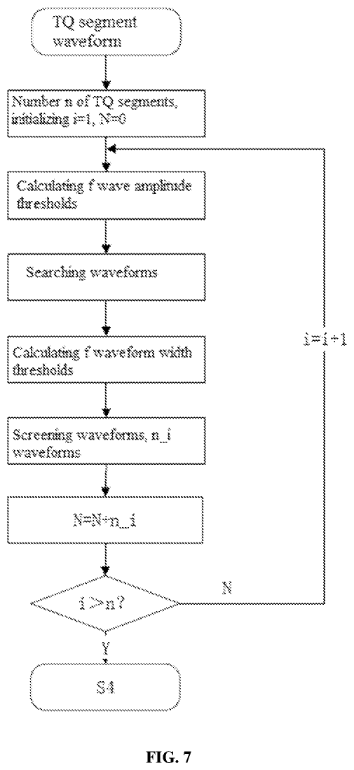

Specifically, as shown in FIG. 6, the fourth model is S4=100 exp(−δ), where S4 is the fourth score, and δ is the coefficient of the fourth model.

Whereinfor the detection module, the step of performing conditional judgment on a ratio of P point amplitude to R point amplitude through a fourth model to obtain a fourth score includes:

in order to determine whether the ratio of P point amplitude to R point amplitude is a normal ratio of P point amplitude to R point amplitude, it needs to judge whether the ratio of P point amplitude to R point amplitude is within the threshold range.

Specifically, if the ratio of P point amplitude to R point amplitude in a heartbeat waveform is within a range of 0.1-0.2, the corresponding heartbeat waveform is determined as a normal PR height ratio and recorded.

A ratio q of the number of heartbeat waveforms with a normal PR height ratio to the number of all heartbeat waveforms is acquired in an ECG signal.

The ratio q is initialized as 0.

It is judged whether q is less than or equal to 0.9. When the ratio q is greater than 0.9, δ is determined as 0.6931.

When the ratio q is less than or equal to 0.9, it is judged whether q is less than or equal to 0.8.

When the ratio q is less than or equal to 0.9 and greater than 0.8, δ is determined as −5.109×q+5.2912.

When the ratio q is less than or equal to 0.8, it is judged whether q is less than or equal to 0.6.

When the ratio q is less than or equal to 0.8 and greater than 0.6, δ is determined as 8.9585×q+8.3708.

When the ratio q is less than or equal to 0.6, it is judged whether q is less than or equal to 0.4.

When the ratio q is less than or equal to 0.6 and greater than 0.4, δ is determined as 2.9957.

When the ratio q is greater than 0.4, δ is determined as 5.

It shall be noted that, among the leads of a normal ECG, lead II has the most obvious P wave. Since the P wave disappears and the f wave of lead II is not obvious, the amplitude of the P wave found at this time is small. Even if there is no P wave, a position that is considered to be the P wave is found; actually, this position is not a P wave, and the amplitude is small. Therefore, the present invention considers that if the ratio of P point amplitude to R point amplitude is within a certain range, it means that it may be a real P wave. A proportion of waveforms with suspected real P wave is used as a detection standard, that is, the detection standard q of the fourth model. The greater the q value is, the more the suspected real P waves are, the smaller the possibility for the disappearance of P waves is, and the smaller the possibility of atrial fibrillation is.

In an embodiment, for the detection module, the step of integrating the first score, the second score, the third score and the fourth score to obtain an integrated score includes:

when the first score S1 is 0, the integrated score S is determined as 0; and

when the first score S1 is not 0, the integrated score S is the difference value between a sum of the first score S1 and the second score S2 and a sum of the third score S3 and the fourth score S4. That is, S=S1+S2−S3−S4.

It shall be noted that, all the scores of the first model to the fourth model can be calculated just based on lead-II ECG signals, and the scores of these four models are first fused. According to the contents recorded in the above-mentioned examples, the greater the values of the first model and the second model are, the greater the degree of absolute unevenness is, and the more likely it is atrial fibrillation. The greater the value of the third model is, the smaller the degree of absolute unevenness is, and the more unlikely it is atrial fibrillation. The greater the value of the fourth model is, the greater the possibility of normal P wave is, and the less likely it is atrial fibrillation.

Therefore in the present invention, the score of the fusion module is obtained by S1+S2−S3−S4 as the final score, that is, S1 and S2 play a role in increasing the suspected degree, and S3 and S4 play a role in reducing the suspected degree. In this way, the scores of the above four models can be balanced to obtain a more accurate result.

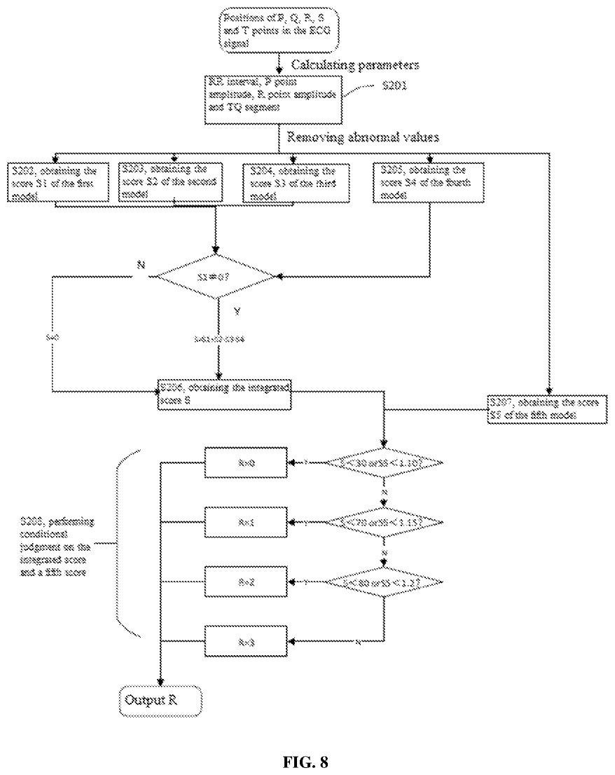

FIG. 7 is a flow chart for acquiring a fifth score by a fifth model provided by an embodiment of the present invention;

As shown in FIG. 7, for the fifth model, it needs to input all TQ segment waveforms between heartbeats within 20 s, including calculating amplitude thresholds, searching waveforms, calculating width thresholds and screening waveforms.

The fifth model is S5=N/n, S5 is a score of the fifth model, n is a total number of TQ segment waveforms involved in an ECG signal, and N is a sum of n_i of TQ segment waveforms involved in the ECG signal.

Wherein for the detection module, the step of determining the fifth score through the fifth model includes:

S101: a total number n of TQ segment waveforms involved in an ECG signal is acquired. It is initialized as i=1, that is, the serial number of the ith TQ segment.

S102: for any TQ segment waveform, an amplitude v_T of the T point and an average amplitude v_TQ of the entire TQ segment are calculated.

S103: in order to search a significant characteristic f wave of atrial fibrillation, an amplitude threshold th_h=v_TQ+(v_T−v_TQ)/40 of the f wave of the current TQ segment waveform is calculated.

S104: the waveforms greater than the amplitude threshold of respective f waveform in each of the TQ segment waveforms are determined.

Specifically, the ith TQ segment is searched to find out the waveforms greater than the amplitude threshold of respective f waveform, recording as a set W_i.

S105: a width of each waveform greater than the amplitude threshold of respective f waveform in each of the TQ segment waveforms is determined, and the waveform max_w with the maximum width in each TQ segment waveform is determined.

Specifically, the width of each of the waveforms in the set W_i is calculated to find out the waveform with the maximum width, and the width is recorded as max_w.

S106: in order to filter the real f wave in W_i, a width threshold th_w of the TQ segment is calculated, and the the width threshold th_w=0.4×max_w of each TQ segment is determined.

S107: a number n_iof f waves in each of the TQ segment waveforms is determined, wherein the f wave of each of the TQ segment waveforms is waveform greater than the amplitude threshold of respective f-waveform and with the width greater than the width threshold.

Specifically, all waveforms in W_i are searched to find out the waveforms with the width greater than th_w, and the number is recorded as n_i.

Then, N=N+n_j is calculated, and it is judged whether the current i value is greater than n. If i>n, go to S108, otherwise set i=i+1, and go back to S102.

S108: wherein the fifth model is S5=N/n, that is, determining that the fifth score is a quotient of a sum off waves in all TQ segment waveforms in the ECG signal to a total number n of the TQ segment waveforms involved in the ECG signal.

It shall be noted that, the present invention has studied all lead signals and found that lead-V1 signals have the most obvious f waves, and thus the present invention collects the lead-V1 signals to facilitate the judgment of f waves. Owing to the relatively small amplitude, f waves usually cannot reflect in the QRS waves and T waves in the prior art. It is relatively gentle between the T wave of a previous heartbeat to the Q wave of a next heartbeat, and thus in order to characterize the appearance of the f wave, the present invention studies the TQ band and searches f waves on the TQ band by the above method. The f waves are searched and counted on each TQ segment, the mean value of a number of f waves in all TQ segments can be scientifically calculated using the above method and used as the result S5 of the fifth model. The greater the S5 is, the greater the possibility for the appearance of f waves, and the more likely to suffer from atrial fibrillation.

In an embodiment, for the detection model, the step of performing conditional judgment on the integrated score S and the fifth score S5 to determine a suspected degree of suffering from atrial fibrillation includes:

When the integrated score S is less than 30 or the fifth score S5 is less than 1.1, it is determined as not suffering from atrial fibrillation.

When the integrated score is greater than or equal to 30 and the fifth score is greater than or equal to 1.1, and meanwhile the integrated score is less than 70, it is determined as mildly suspected atrial fibrillation.

When the integrated score is greater than or equal to 30 and the fifth score is greater than or equal to 1.1, and meanwhile the fifth score is less than 1.15, it is determined as mildly suspected atrial fibrillation.

When the integrated score is greater than or equal to 70 and the fifth score is greater than or equal to 1.15, if the integrated score is less than 80, it is determined as suspected atrial fibrillation.

When the integrated score is greater than or equal to 70 and the fifth score is greater than or equal to 1.15, if the fifth score is less than 1.2, it is determined as suspected atrial fibrillation.

When the integrated score is greater than or equal to 80 and the fifth score is greater than or equal to 1.2, it is determined as suffering from atrial fibrillation.

Or for convenience of showing the detection results, it could be set that if S<30 or S5<1.1, set the result R=0. Otherwise, if S<70 or S5<1.15, set the result R=1. Otherwise, if S<80 or S5<1.2, set the result R=2. Otherwise, set the result R=3. The final result is that R=0 represents no atrial fibrillation, R=1 represents mildly suspected atrial fibrillation, R=2 represents suspected atrial fibrillation, and R=3 represents suffering from atrial fibrillation.

In an embodiment, the detection model also includes an alarm module connected with the detection model, and the alarm module is configured for giving an alarm when mildly suspected atrial fibrillation and atrial fibrillation is detected by the detection model.

Specifically, when the detection module determines the R value, it sends a control signal to the alarm module to control the alarm module to give an alarm.

For example, when the detection module determined that R=1, it sent a first control signal to the alarm module to control the alarm module to send a first alarm; for example, when the detection module determined that R=2, it sent a second control signal to the alarm module to control the alarm module to give a second alarm; and when the detection module determined that R=3, it sent a third control signal to the alarm module to control the alarm module to give a third alarm.

In a specific embodiment, it can also be set that when the detection module determined that the integrated score is greater than or equal to 30 and the fifth score is greater than or equal to 1.1, the integrated score is less than 70, the integrated score is greater than or equal to 30 and the fifth score is greater than or equal to 1.1, and the fifth score is less than 1.15, the detection module sends the first control signal to the alarm module, and a first control signal is configured for instructing the alarm module to send a first alarm.

Or when the detection module determined that the integrated score is greater than or equal to 70 and the fifth score is greater than or equal to 1.15, if the integrated score is less than 80; or the integrated score is greater than or equal to 70 or the fifth score is greater than or equal to 1.15, and the fifth score is less than 1.2, the detection module sends a second control signal to the alarm module, and the second control signal is configured for instructing the alarm module to send a second alarm.

When the detection module determined that the integrated score is greater than or equal to 80 or the fifth score is greater than or equal to 1.2, the detection module sends a third control signal to the alarm module, and the third control signal is configured for instructing the alarm module to send a third alarm.

In the embodiment, the alarm module includes, for example, one or more buzzers, wherein the first alarm, the second alarm and the third alarm can be sound alarms.

Preferably, the sound emitted by the first alarm, the second alarm and the third alarm lasts for different lengths of time. For example, the sound emitted by the first alarm lasts for 1-5 s, preferably 3-5 s, the sound emitted by the second alarm lasts for 6-10 s, preferably 8-10 s, and the sound emitted by the third alarm lasts for 11-15 s, preferably 13-15 s.

Preferably, the sounds emitted by the first alarm, the second alarm and the third alarm are at different frequencies, or the sounds emitted by the first alarm, the second alarm and the third alarm have different amplitudes to distinguish three different severity of disease.

It can be understood that, the first alarm, the second alarm and the third alarm can be given by the same buzzer, or the three alarms can be given by three groups of buzzers in one-to-one correspondence.

It shall be noted that, although the integrated scores of the first four models can obtain the detection results, it is unable and not sufficient to fully determine the degree of suffering from atrial fibrillation because the f wave is not judged, and thus the integrated score is fused with the score of the fifth model. The greater the integrated score S is, the greater the suspected degree of atrial fibrillation is. The greater the score S5 of the fifth model, the greater the suspected degree of atrial fibrillation is. Therefore, the present invention sets multiple judgment intervals for the two scores to obtain different levels of disease by different layers; compared with directly indicating the judgment whether suffering from atrial fibrillation, the degree of disease can be obtained, so that it can better reflect the suspected degree of atrial fibrillation movement, for convenience of timely treatment. In addition, compared with the existing judgment methods that require a longer ECG signal or have a delay in diagnosis, the present invention only needs to collect an ECG signal of 20 s, so that realtime judgment on atrial fibrillation can be achieved.

FIG. 8 is a flow chart of an atrial fibrillation detection method provided by an embodiment of the present invention.

As shown in FIG. 8, the method includes S201-S207.

In a preferable embodiment, prior to S201, ECG signals are firstly collected every preset time and the ECG signal includes a lead-II ECG signal and a lead-V1 ECG signal.

Specifically, an original ECG signal is collected from a human body surface by portable hardware, and the original ECG signal is processed to eliminate interference to obtain a usable EGC signal.

Specifically, an acquired ECG signal is decomposed into 8 layers using a db6 wavelet. A wavelet coefficient obtained by decomposition is processed by a soft threshold method to obtain a modified wavelet coefficient. The signal is then reconstructed by the modified wavelet coefficient to obtain a usable ECG signal.

S201: positions of P, Q, R, S and T points of all heartbeats in an ECG signal acquired in a preset time are identified, and a RR interval, a P point amplitude, an R point amplitude and a TQ segment waveform of each heartbeat are determined according to the positions of the P, Q, R, S and T points.

Specifically, the step of identifying positions of P, Q, R, S and T points of all heartbeats in an ECG signal acquired in a preset time includes: detecting the main characteristic points of the ECG signal based on a biorthogonal wavelet and a first-order difference.

Further specifically, a QRS complex in a lead-II ECG signal is detected using a B-spline biorthogonal wavelet to determine the positions of the Q, R and S points.

The lead-II ECG signal is identified using the first-order difference to obtain the positions of the P and T points.

Wherein the step of determining a RR interval, a P point amplitude, an R point amplitude and a TQ segment waveform of each heartbeat according to the positions of the P, Q, R, S and T includes:

obtaining waveforms of all TQ segments based on the positions of the T point and the nearest Q point after the T point and the lead-V1 ECG;

obtaining RR intervals between heartbeats from the positions of all R points and the lead-II ECG signal;

obtaining P point amplitudes of the heartbeats from the positions of all P points and the lead-II ECG signal; and

obtaining R point amplitudes of the heartbeats from the positions of all R points and the lead-II ECG signal.

In a preferable embodiment, S101 also includes: removing a RR interval that is greater than 0.5 times the mean value and less than 1.6 times the mean value within a preset time (20 s).

Specifically, a mean value of all RR intervals is calculated. Then each RR interval is judged whether it is greater than 0.5 times the mean value and less than 1.6 times the mean value. If the condition is not satisfied, the RR interval is deemed as an abnormal value and eliminated.

S202: conditional judgment is performed on an extremal ratio of the RR intervals through a first model to obtain a first score.

For the first model, it needs to input a maximum-to-minimum ratio of each RR interval, determine the maximum values and the minimum values of all RR intervals, determine the maximum-to-minimum ratios of the RR intervals, determine a range of the maximum-to-minimum ratios of the RR intervals, responds to the range of the maximum-to-minimum ratios of the RR intervals, and calculate a coefficient to calculate a score of the first model base on the coefficient.

Specifically, the first model is S1=100 exp(−α), where S1 is the first score, and α is a coefficient of the first model.

For the detection module, the step of performing conditional judgment on an extremal ratio of the RR intervals through a first model to obtain a first score includes: a ratio r of a duration of a maximum RR interval to that of a minimum RR interval is obtained according to all inputted RR intervals.

It is judged whether ratio r is less than or equal to 3.0. When the ratio r is greater than 3, the coefficient α is determined as 0.6931.

Then, it is judged whether the ratio r is less than or equal to 2.1. when the ratio r is less than or equal to 3 and greater than 2.1, α is determined as −0.5677×r+2.3962.

Then, it is judged whether the ratio r is less than or equal to 1.9. When the ratio r is less than or equal to 2.1 and greater than 1.9, α is determined as 1.204.

Then, it is judged whether the ratio r is less than or equal to 1.1. When the ratio r is less than or equal to 1.9 and greater than 1.1, α is determined as −4.745×r+10.2195.

When the ratio r is less than or equal to 1.1, α is determined as 5.

S203: conditional judgment is performed on a ratio of a number of the RR intervals with a deviation value exceeding a standard deviation to a number of all the RR intervals in the ECG signal acquired in the preset time through a second model to obtain a second score.

For the second model, it is needs to input a proportion of RR intervals with a deviation value exceeding a standard deviation, determine a mean value of all RR intervals and the standard deviation, determine whether the deviation of each RR interval from the mean value exceeds the standard deviation, determine a ratio p of the number of the RR intervals with the deviation value exceeding the standard deviation to the number of all the RR intervals, respond to a numerical range of the ratio p to obtain a coefficient value of the second model, and calculate a score of the second model base on the coefficient.

Specifically, the second model is S2=100 exp(−β), where S2 is the second score, and β is the coefficient of the second model.

Wherein the step of performing conditional judgment on a ratio of a number of the RR intervals with a deviation value exceeding a standard deviation to a number of all the RR intervals in the ECG signal acquired in the preset time through a second model to obtain a second score includes:

according to all the inputted RR intervals, obtaining the mean value of RR intervals and the deviation value of each RR interval from the mean value; and

determining the ratio p of the number of the RR intervals with the deviation exceeding the standard deviation to the number of all the RR intervals.

First, it is judged whether the ratio p is less than or equal to 0.45, and when the ratio p is greater than 0.45, β is determined as 1.204.

Whether the ratio p is less than or equal to 0.45, it is judged whether p is less than or equal to 0.35.

When the ratio p is less than or equal to 0.45 and greater than 0.35, β is determined as −10.896×p+6.1477.

When the ratio p is less than or equal to 0.35, it is judged whether the ratio p is less than or equal to 0.25.

When the ratio p is less than or equal to 0.35 and greater than 0.25, β is determined as −26.974×p+11.7435.

When the ratio p is less than or equal to 0.25, β is determined as 5.

S204: conditional judgment is performed on a number of the RR interval groups similar to other arrhythmia in the ECG signal acquired in the preset time through a third model to obtain a third score.

Wherein for the third model, it needs to input a number of RR interval groups conforming to complete compensatory pause and approximate to the type of premature beats, process 4 consecutive RR intervals as one RR interval group to determine whether each RR interval group conforms to complete compensatory pause, determine whether each RR interval group is approximate to the type of premature beats, determine a number of RR interval groups conforming to complete compensatory pause and approximate to the type of premature beats, determine a range of the number of RR interval groups conforming to complete compensatory pause and approximate to the type of premature beats, respond to the range of the number, calculate a coefficient value, and calculate a score of the third model from the coefficient.

Specifically, the third model is S3=100 exp(−γ), where S3 is the third score, and y is the coefficient of the third model.

Wherein for the detection module, the step of performing conditional judgment on a number of the RR interval groups similar to other arrhythmia through a third model to obtain a third score includes:

four continuous RR intervals are taken as a RR interval group according to a time sequence, a sum of a second RR interval and a third RR interval is compared with a mean value of RR intervals;

if the sum of the second RR interval and the third RR interval is less than 2.2 times the mean value of RR intervals and greater than 1.1 times the mean value of RR intervals, it meets the judgment condition 1. Next, the four RR intervals are compared to determine whether they meet the judgment condition 2 for being similar to other arrhythmia If the first RR interval is greater than the second RR interval, the third RR interval is greater than the second RR interval and the fourth RR interval, it is determined to meet the judgment condition 2. The RR interval groups meeting both judgment conditions at the same time are those similar to other arrhythmia, and a number of the RR interval groups is recorded as n.

That is, if the sum of the second RR interval and the third RR interval is less than 2.2 times the mean value of RR intervals and greater than 1.1 times the mean value of RR intervals, the first RR interval is greater than the second RR interval, and the third RR interval is greater than the second RR interval and the fourth RR interval, the RR interval group is determined as a RR interval group similar to other arrhythmia

The coefficient γ is initialized as 0, and then the value of n is judged.

It is firstly judged whether n is less than or equal to 4. When the number n of RR interval groups similar to other arrhythmia is greater than 4, γ is determined to be 0.6931.

When n is less than or equal to 4, it is judged whether n is less than or equal to 3.

When the number n is less than or equal to 4 and greater than 3, γ is determined as 1.204.

When n is less than or equal to 3, it is judged whether n is less than or equal to 2.

When the number n is less than or equal to 3 and greater than 2, γ is determined as 1.8971.

When the number n is less than or equal to 2 and greater than 1, γ is determined as 2.9957.

When the number n is less than or equal to 1, γ is determined as 5.

S205: conditional judgment is performed on a ratio of a number of heartbeat waveforms with a normal PR height ratio to a number of normal heartbeat waveforms in the ECG signal through a fourth model to obtain a fourth score.

For the fourth model, it needs to input P point amplitudes and R point amplitudes of all waveforms within 20 s. Then, it needs to determine the PR height ratios of all waveforms, determine whether the PR height ratio of each waveform is within a threshold range, determine a proportion of the waveforms with the PR height ratio within the threshold range, determine a range of the proportion of the waveforms with the PR height ratio within the threshold range, respond to the range of the proportion, calculate a coefficient value, and calculate a score of the fourth model from the coefficient.

Specifically, the fourth model is S4=100 exp(−δ), where S4 is the fourth score, and δ is the coefficient of the fourth model.

Wherein for the detection module, the step of performing conditional judgment on a ratio of P point amplitude to R point amplitude through a fourth model to obtain a fourth score includes:

in order to determine whether the ratio of P point amplitude to R point amplitude is a normal ratio of P point amplitude to R point amplitude, it needs to judge whether the ratio of P point amplitude to R point amplitude is within the threshold range.

Specifically, if the ratio of P point amplitude to R point amplitude is within a range of 0.1-0.2, the corresponding waveform is determined as a normal PR height ratio and recorded.

The ratio q of the number of waveforms with the ratio of P point amplitude to R point amplitude within 0.1-0.2 to the number of all waveforms in the preset time (20 s) is acquired.

The ratio q is initialized as 0.

It is judged whether q is less than or equal to 0.9. When the ratio q is greater than 0.9, δ is determined as 0.6931.

When the ratio q is less than or equal to 0.9, it is judged whether q is less than or equal to 0.8.

When the ratio q is less than or equal to 0.9 and greater than 0.8, δ is determined as −5.109×q+5.2912.

When the ratio q is less than or equal to 0.8, it is judged whether q is less than or equal to 0.6.

When the ratio q is less than or equal to 0.8 and greater than 0.6, δ is determined as 8.9585×q+8.3708.

When the ratio q is less than or equal to 0.6, it is judged whether q is less than or equal to 0.4.

When the ratio q is less than or equal to 0.6 and greater than 0.4, δ is determined as 2.9957.

When the ratio q is greater than 0.4, δ is determined as 5.

S206: the first score, the second score, the third score and the fourth score are integrated to obtain an integrated score.

Specifically, when the first score S1 is 0, the integrated score S is determined as 0; and

when the first score S1 is not 0, the integrated score S is the difference value between a sum of the first score S1 and the second score S2 and a sum of the third score S3 and the fourth score S4. That is, S=S1+S2−S3−S4.

S207: a fifth score is obtained according to the fifth model. Wherein the fifth score is a quotient of a sum of f waves in all TQ segment waveforms in the ECG signal acquired in the preset time to a total number n of the TQ segment waveforms involved in the ECG signal.

Wherein for the fifth model, it needs to input all TQ segment waveforms between heartbeats within 20 s, including calculating amplitude thresholds, searching waveforms, calculating width thresholds and screening waveforms.

The fifth model is S5=N/n, S5 is the score of the fifth model, n is the total number of TQ segment waveforms involved in the ECG signal, and N is a sum of n_i of TQ segment waveforms involved in the ECG signal.

Wherein for the detection module, the step of determining the fifth score through the fifth model includes S101-S108:

S101: the total number n of TQ segment waveforms involved in the ECG signal is acquired. It is initialized as i=1, that is, the serial number of the ith TQ segment.

S102: for any TQ segment waveform, an amplitude v_T of the T point and an average amplitude v_TQ of the entire TQ segment are calculated.

S103: in order to search a significant characteristic f wave of atrial fibrillation, an amplitude threshold th_h=v_TQ+(v_T−v_TQ)/40 of the f wave of the current TQ segment waveform is calculated.

S104: the waveforms greater than the amplitude threshold of respective f waveform in each of the TQ segment waveforms are determined.

Specifically, the ith TQ segment is searched to find out the waveforms greater than the amplitude threshold of respective f waveform, recording as a set W_i.

S105: a width of each waveform greater than the amplitude threshold of respective f waveform in each of the TQ segment waveforms is calculated, and the waveform max_w with the maximum width is determined.

Specifically, the width of each of the waveforms in the set W_i is calculated to find out the waveform with the maximum width, and the width is recorded as max_w.

S106: in order to filter the real f wave in W_i, a width threshold th_w of the TQ segment is calculated, and the the width threshold th_w=0.4×max_w of each TQ segment is determined.

S107: a number n_i of waveforms greater than the amplitude threshold of respective f-waveform and with the width greater than the width threshold in each of the TQ segment waveforms is determined.

Specifically, all waveforms in W_i are searched to find out the waveforms with the width greater than th_w, and the number is recorded as n_i.

Then, N=N+n_j is calculated, and it is judged whether the current i value is greater than n. If i>n, go to S108, otherwise set i=i+1, and go back to S102.

S108: wherein the fifth model is S5=N/n, that is, determining that the fifth score is a quotient of a sum of n_i of all TQ segment waveforms in the ECG signal to a total number of the TQ segment waveforms involved in the ECG signal.

It shall be noted that, S202-S205 are not in a sequential order, and they can be performed separately or according to the existing order. Alternatively, S202-S205 and S207 can be performed simultaneously to obtain the first score to fifth score, respectively. Alternatively, S206 and S207 are not in a sequential order.

S208 “performing conditional judgment on the integrated score and a fifth score to determine a suspected degree of suffering from atrial fibrillation”includes:

When the integrated score S is less than 30 or the fifth score S5 is less than 1.1, it is determined as not suffering from atrial fibrillation.

When the integrated score is greater than or equal to 30 and the fifth score is greater than or equal to 1.1, and meanwhile the integrated score is less than 70, it is determined as mildly suspected atrial fibrillation;

When the integrated score is greater than or equal to 30 and the fifth score is greater than or equal to 1.1, and meanwhile the fifth score is less than 1.15, it is determined as mildly suspected atrial fibrillation.

When the integrated score is greater than or equal to 70 and the fifth score is greater than or equal to 1.15, if the integrated score is less than 80, it is determined as suspected atrial fibrillation.

When the integrated score is greater than or equal to 70 and the fifth score is greater than or equal to 1.15, if the fifth score is less than 1.2, it is determined as suspected atrial fibrillation.

When the integrated score is greater than or equal to 80 and the fifth score is greater than or equal to 1.2, it is determined as suffering from atrial fibrillation.

In an embodiment, it also included S209 after S208: sending different types of alarm signals according to different suspected degree of atrial fibrillation.

An embodiment of the present invention provides an atrial fibrillation detection system, including a memory and one or more processors; wherein the memory is connected with the one or more processors, and instructions executable for the one or more processors are stored in the memory; the instructions are executed by the one or more processors to make the one or more processors execute the above-mentioned method.

An embodiment of the present invention provides a computer readable storage medium on which computer executable instructions are stored. When the computer executable instructions are executed, the above-mentioned method can be performed by operation.

It shall be understood that the above embodiment described is used only for stating embodiments or explaining the principle of the present invention, rather than limiting the present invention. Therefore, any modification, equivalent alternation or improvement without deviating from the spirit and scope of the present application will fall within the protection scope of the present application. In addition, the claims of the present invention are intended to cover all the changes and modifications within the scope and boundary, as well as the equivalent forms of the scope and boundary.

What is claimed is:

1. An atrial fibrillation detection device, comprising:
an ECG signal processing module, configured for identifying positions of P, Q, R, S and T points of all heartbeats in an ECG signal acquired in a preset time and determining a RR interval, a P point amplitude, an R point amplitude and a TQ segment waveform of each heartbeat according to the positions of the P, Q, R, S and T points; and
a detection module, configured for performing conditional judgment on an extremal ratio of RR intervals in the ECG signal through a first model to obtain a first score, performing conditional judgment on a number of the RR intervals with a deviation value exceeding a standard deviation and a ratio of all the RR intervals in the ECG signal through a second model to obtain a second score, performing conditional judgment on a number of the RR interval groups similar to other arrhythmia in the ECG signal through a third model to obtain a third score, performing conditional judgment on a ratio of a number of heartbeat waveforms with a normal PR height ratio to a number of normal heartbeat waveforms in the ECG signal through a fourth model to obtain a fourth score, integrating the first score, the second score, the third score and the fourth score to obtain an integrated score, and performing conditional judgment on the integrated score and a fifth score to determine a suspected degree of suffering from atrial fibrillation, wherein the fifth score is a quotient of a total number of f waves in all the TQ segment waveforms in the ECG signal to a total number of the TQ segment waveforms involved in the ECG signal, and wherein the f waves in each of the TQ segment waveforms are the waveforms greater than an amplitude threshold of respective f waveform and with a width greater than a width threshold.

2. The detection device of claim 1, wherein for the detection module, the step of performing conditional judgment on an extremal ratio of the RR intervals in the ECG signal through a first model to obtain a first score, comprises:
the first model is:
S1=100 exp(−α), where S1 is the first score, and α is a coefficient of the first model;
a ratio r of a length of a maximum RR interval to that of a minimum RR interval is obtained according to all RR intervals in the ECG signal;
when the ratio r is greater than 3, the coefficient is determined as 0.6931;
when the ratio r is less than or equal to 3 and greater than 2.1, α is determined as −0.5677×r+2.3962;
when the ratio r is less than or equal to 2.1 and greater than 1.9, α is determined as 1.204;
when the ratio r is less than or equal to 1.9 and greater than 1.1, α is determined as −4.745×r+10.2195; and
when the ratio r is less than or equal to 1.1, α is determined as 5.

3. The detection device of claim 1, wherein for the detection module, the step of performing conditional judgment on a ratio of a number of the RR intervals with a deviation value exceeding a standard deviation in the ECG signal to a number of all the RR intervals through a second model to obtain a second score, comprises:
the second model is
S2=100 exp(β), where S2 is the second score, and β is a coefficient of the second model;
the mean value of the RR intervals and a deviation value of each RR interval from the mean value are obtained according to all the RR intervals in the ECG signal;
the ratio p of the number of the RR intervals with the deviation value exceeding the standard deviation to the number of all the RR intervals is determined;
when the ratio p is greater than 0.45, β is determined as 1.204;
when the ratio p is less than or equal to 0.45 and greater than 0.35, β is determined as −10.896×p+6.1477;
when the ratio p is less than or equal to 0.35 and greater than 0.25, β is determined as −26.974×p+11.7435; and
when the ratio p is less than or equal to 0.25, β is determined as 5.

4. The detection device of claim 1, wherein for the detection module, the step of performing conditional judgment on a number of the RR interval groups similar to other arrhythmia in the ECG signal through a third model to obtain a third score, comprises:
the third model is
S3=100 exp(−γ), where S3 is the third score, and γ is a coefficient of the third model;
four continuous RR intervals in all the RR intervals in the ECG signal are taken as a RR interval group according to a time sequence;
a sum of a second RR interval and a third RR interval in each RR interval group is compared with a mean value of the RR intervals in the ECG signal within the preset time;
if the sum of the second RR interval and the third RR interval is less than 2.2 times the mean value of the RR intervals and greater than 1.1 times the mean value of the RR intervals, the first RR interval is greater than the second RR interval, and the third RR interval is greater than the second RR interval and the fourth RR interval, the RR interval group is determined as a RR interval group similar to other arrhythmia;
when the number of the RR interval groups similar to other arrhythmia is greater than 4, γ is determined as 0.6931;
when the number of the RR interval groups similar to other arrhythmia is less than or equal to 4 and greater than 3, γ is determined as 1.204;
when the number of the RR interval groups similar to other arrhythmia is less than or equal to 3 and greater than 2, γ is determined as 1.8971;
when the number of the RR interval groups similar to other arrhythmia is less than or equal to 2 and greater than 1, γ is determined as 2.9957; and
when the number of the RR interval groups similar to other arrhythmia is less than or equal to 1, γ is determined as 5.

5. The detection device of claim 1, wherein
for the detection module, the step of performing conditional judgment on a ratio of a number of heartbeat waveforms with a normal PR height ratio to a number of all heartbeat waveforms in the ECG signal through a fourth model to obtain a fourth score, comprises:
the fourth model is
S4=100 exp(−δ), where S4 is the fourth score, and δ is a coefficient of the fourth model;
if the ratio of P point amplitude to R point amplitude in a heartbeat waveform is
within a range of 0.1-0.2, the corresponding heartbeat waveform is determined as a normal PR height ratio;
a ratio q of the number of heartbeat waveforms with a normal PR height ratio to the number of all heartbeat waveforms in the ECG signal is acquired;
when the ratio q is greater than 0.9, δ is determined as 0.6931;
when the ratio q is less than or equal to 0.9 and greater than 0.8, δ is determined as −5.109×q+5.2912;
when the ratio q is less than or equal to 0.8 and greater than 0.6, δ is determined as −8.9585×q+8.3708;
when the ratio q is less than or equal to 0.6 and greater than 0.4, δ is determined as 2.9957; and
when the ratio q is greater than 0.4, δ is determined as 5.

6. The detection device of any of the claim 1, wherein for the detection module, the step of determining a fifth score comprises:
acquiring a total number n of TQ segment waveforms involved in the ECG signal;
for any TQ segment waveform, calculating an amplitude v_T of the T point and an average amplitude v_TQ of the entire TQ segment;
setting an amplitude threshold th_h=v_TQ+(v_T−v_TQ)/40 of the f wave of the current TQ segment waveform;
determining the waveforms greater than the amplitude threshold th_h of respective f waveform in each of the TQ segment;
calculating a width of each waveform greater than the amplitude threshold of respective f waveform in each of the TQ segment waveforms, and determining the waveform max_w with the maximum width in each TQ segment waveform;
determining the width threshold th_w of each TQ segment waveform as 0.4×max_w; and
determining a number n_iof f waves in each of the TQ segment waveforms, wherein the f wave of each of the TQ segment waveforms is waveform greater than the amplitude threshold of respective f-waveform and with the width greater than the width threshold;
the fifth score is a quotient of a sum of f waves in all TQ segment waveforms in the ECG signal to a total number n of the TQ segment waveforms involved in the ECG signal.

7. The detection device of claim 6, wherein for the detection module, the step of integrating the first score, the second score, the third score and the fourth score to obtain an integrated score, comprises:
when the first score is 0, the integrated score is determined as 0; and
when the first score is not 0, the integrated score is the difference value between a sum of the first score and the second score and a sum of the third score and the fourth score.

8. The detection device of claim 7, wherein for the detection module, the step of performing conditional judgment on the integrated score and the fifth score to determine a suspected degree of suffering from atrial fibrillation, comprises:
when the integrated score is less than 30 or the fifth score is less than 1.1, it is determined as not suffering from atrial fibrillation;
when the integrated score is greater than or equal to 30 and the fifth score is greater than or equal to 1.1, and meanwhile the integrated score is less than 70, it is determined as mildly suspected atrial fibrillation;
when the integrated score is greater than or equal to 30 and the fifth score is greater than or equal to 1.1, and meanwhile the fifth score is less than 1.15, it is determined as mildly suspected atrial fibrillation;
when the integrated score is greater than or equal to 70 and the fifth score is greater than or equal to 1.15, if the integrated score is less than 80, it is determined as suspected atrial fibrillation;
when the integrated score is greater than or equal to 70 and the fifth score is greater than or equal to 1.15, if the fifth score is less than 1.2, it is determined as suspected atrial fibrillation; and
when the integrated score is greater than or equal to 80 and the fifth score is greater than or equal to 1.2, it is determined as suffering from atrial fibrillation.

9. The detection device of claim 6, also comprising:
a signal acquisition module, configured for collecting ECG signals every preset time, wherein the ECG signal comprises a lead-II ECG signal and a lead-V1 ECG signal;
the signal acquisition module is configured for detecting a QRS complex in a lead- II ECG signal using a B-spline biorthogonal wavelet to determine the positions of the Q, R and S points;
the lead-II ECG signal is identified using the first-order difference to obtain the positions of the P and T points;
waveforms of all TQ segments are obtained based on the positions of the T point and the nearest Q point after the T point and the lead-V1 ECG;
RR intervals between heartbeats are obtained from the positions of all R points;
P point amplitudes of the heartbeats are obtained from the positions of all P points and the lead-II ECG signal; and
R point amplitudes of the heartbeats are obtained from the positions of all R points and the lead-II ECG signal.

10. The method of claim 1, wherein the signal processing module is also configured for removing a RR interval that is greater than 0.5 times the mean value and less than 1.6 times the mean value.

11. An atrial fibrillation detection method, comprising the following steps:
identifying positions of Q, R, S and T points of all heartbeats in an ECG signal acquired in a preset time, and determining a RR interval, a P point amplitude, an R point amplitude and a TQ segment waveform of each heartbeat according to the positions of the P, Q, R, S and T positions;
performing conditional judgment on an extremal ratio of the RR intervals in the ECG signal through a first model to obtain a first score;
performing conditional judgment on a ratio of a number of RR intervals with a deviation value exceeding a standard deviation to a number of all the RR intervals in the ECG signal through a second model to obtain a second score;
performing conditional judgment on a number of the RR interval groups similar to other arrhythmia in the ECG signal through a third model to obtain a third score;
performing conditional judgment on a ratio of a number of heartbeat waveforms with a normal PR height ratio to a number of all heartbeat waveforms in the ECG signal through a fourth model to obtain a fourth score;
integrating the first score, the second score, the third score and the fourth score to obtain an integrated score; and
performing conditional judgment on the integrated score and a fifth score to determine a suspected degree of suffering from atrial fibrillation, wherein the fifth score is a quotient of a total number of f waves in all the TQ segment waveforms in the ECG signal to a total number of the TQ segment waveforms involved in the ECG signal;
wherein the f wave of each of the TQ segment waveforms is waveform greater than the amplitude threshold of respective f-waveform and with the width greater than the width threshold.

12. The method of claim 11, wherein the step of performing conditional judgment on an extremal ratio of the RR intervals in the ECG signal through a first model to obtain a first score comprises:
the first model is
S1=100 exp(−α), where S1 is the first score, and α is a coefficient of the first model;
a ratio r of a length of a maximum RR interval to that of a minimum RR interval is obtained according to all RR intervals in the ECG signal;
when the ratio r is greater than 3, the coefficient is determined as 0.6931;
when the ratio r is less than or equal to 3 and greater than 2.1, α is determined as −0.5677×r+2.3962;
when the ratio r is less than or equal to 2.1 and greater than 1.9, α is determined as 1.204;
when the ratio r is less than or equal to 1.9 and greater than 1.1, α is determined as −4.745×r+10.2195; and
when the ratio r is less than or equal to 1.1, α is determined as 5;
wherein the step of performing conditional judgment on a ratio of a number of RR intervals with a deviation value exceeding a standard deviation to a number of all the RR intervals in the ECG signal through a second model to obtain a second score comprises:
the second model is
S2=100 exp(β), where S2 is the second score, and β is a coefficient of the second model;
the mean value of the RR intervals and a deviation value of each RR interval from the mean value are obtained according to all the RR intervals in the ECG signal;
the ratio p of the number of the RR intervals with the deviation value exceeding the standard deviation to the number of all the RR intervals is determined;
when the ratio p is greater than 0.45, β is determined as 1.204;
when the ratio p is less than or equal to 0.45 and greater than 0.35, β is determined as −10.896×p+6.1477;
when the ratio p is less than or equal to 0.35 and greater than 0.25, β is determined as −26.974×p+11.7435; and
when the ratio p is less than or equal to 0.25, β is determined as 5.

13. The method of claim 11, wherein the step of performing conditional judgment on a number of the RR interval groups similar to other arrhythmia in the ECG signal through a third model to obtain a third score comprises:
the third model is
S3=100 exp(−γ), where S3 is the third score, and γ is a coefficient of the third model;
four continuous RR intervals in all the RR intervals in the ECG signal are taken as a RR interval group according to a time sequence;
a sum of a second RR interval and a third RR interval in each RR interval group is compared with a mean value of the RR intervals in the ECG signal within the preset time;
if the sum of the second RR interval and the third RR interval is less than 2.2 times the mean value of the RR intervals and greater than 1.1 times the mean value of the RR intervals, the first RR interval is greater than the second RR interval, and the third RR interval is greater than the second RR interval and the fourth RR interval, the RR interval group is determined as a RR interval group similar to other arrhythmia;
when the number of the RR interval groups similar to other arrhythmia is greater than 4, γ is determined as 0.6931;
when the number of the RR interval groups similar to other arrhythmia is less than or equal to 4 and greater than 3, γ is determined as 1.204;
when the number of the RR interval groups similar to other arrhythmia is less than or equal to 3 and greater than 2, γ is determined as 1.8971;
when the number of the RR interval groups similar to other arrhythmia is less than or equal to 2 and greater than 1, γ is determined as 2.9957; and
when the number of the RR interval groups similar to other arrhythmia is less than 1, γ is determined as 5;
wherein the step of performing conditional judgment on a ratio of a number of heartbeat waveforms with a normal PR height ratio to a number of all heartbeat waveforms in the ECG signal through a fourth model to obtain a fourth score comprises:
the fourth model is
S4=100 exp(−δ), where S4 is the fourth score, and δ is a coefficient of the fourth model;
if the ratio of P point amplitude to R point amplitude in a heartbeat waveform is within a range of 0.1-0.2, the corresponding heartbeat waveform is determined as a normal PR height ratio;
a ratio q of the number of heartbeat waveforms with a normal PR height ratio to the number of all heartbeat waveforms in the ECG signal is acquired;
when the ratio q is greater than 0.9, δ is determined as 0.6931;
when the ratio q is less than or equal to 0.9 and greater than 0.8, δ is determined as −5.109×q+5.2912;
when the ratio q is less than or equal to 0.8 and greater than 0.6, δ is determined as −8.9585×q+8.3708;
when the ratio q is less than or equal to 0.6 and greater than 0.4, δ is determined as 2.9957; and
when the ratio q is greater than 0.4, δ is determined as 5.

14. The method of claim 11, wherein the step of determining a fifth score comprises:
acquiring a total number n of TQ segment waveforms involved in the ECG signal;
for any TQ segment waveform, calculating an amplitude v_T of the T point and an average amplitude v_TQ of the entire TQ segment;
setting an amplitude threshold th_h=v_TQ+(v_T−v_TQ)/40 of the f wave of the current TQ segment waveform;
determining the waveforms greater than the amplitude threshold th_h of respective f waveform in each of the TQ segment;
calculating a width of each waveform greater than the amplitude threshold of respective f waveform in each of the TQ segment waveforms, and determining the waveform max_w with the maximum width in each TQ segment waveform;
determining the width threshold th_w of each TQ segment waveform as 0.4×max_w; and
determining a number n_iof f waves in each of the TQ segment waveforms, wherein the f wave of each of the TQ segment waveforms is waveform greater than the amplitude threshold of respective f-waveform and with the width greater than the width threshold;
the fifth score is a quotient of a sum of f waves in all TQ segment waveforms in the ECG signal to a total numbern of the TQ segment waveforms involved in the ECG signal.

15. The method of claim 14, wherein the step of integrating the first score, the second score, the third score and the fourth score to obtain an integrated score comprises:
when the first score is 0, the integrated score is determined as 0; and
when the first score is not 0, the integrated score is the difference value between a sum of the first score and the second score and a sum of the third score and the fourth score.

16. The method of claim 15, wherein the step of performing conditional judgment on the integrated score and the fifth score to determine a suspected degree of suffering from atrial fibrillation comprises:
when the integrated score is less than 30 or the fifth score is less than 1.1, it is determined as not suffering from atrial fibrillation;
when the integrated score is greater than or equal to 30 and the fifth score is greater than or equal to 1.1, and meanwhile the integrated score is less than 70, it is determined as mildly suspected atrial fibrillation;
when the integrated score is greater than or equal to 30 and the fifth score is greater than or equal to 1.1, and meanwhile the fifth score is less than 1.15, it is determined as mildly suspected atrial fibrillation;
when the integrated score is greater than or equal to 70 and the fifth score is greater than or equal to 1.15, if the integrated score is less than 80, it is determined as suspected atrial fibrillation;
when the integrated score is greater than or equal to 70 and the fifth score is greater than or equal to 1.15, if the fifth score is less than 1.2, it is determined as suspected atrial fibrillation; and
when the integrated score is greater than or equal to 80 and the fifth score is greater than or equal to 1.2, it is determined as suffering from atrial fibrillation.

17. The method of claim 11, also comprising the following steps:
before identifying positions of P, Q, R, S and T points of all heartbeats in an ECG signal, it also comprises the following step:
collecting ECG signals every preset time, wherein the ECG signal includes a lead-II ECG signal and a lead-V1 ECG signal;
the step of identifying positions of Q, R, S and T points of all heartbeats in an ECG signal acquired in a preset time comprises the following steps:
identifying the lead-II ECG signal using the first-order difference to obtain the positions of the P and T points;
obtaining waveforms of all TQ segments based on the positions of the T point and the nearest Q point after the T point and the lead-V1 ECG;
obtaining RR intervals between heartbeats from the positions of all R points;
obtaining P point amplitudes of the heartbeats from the positions of all P points and the lead-II ECG signal; and
obtaining R point amplitudes of the heartbeats from the positions of all R points and the lead-II ECG signal.

18. The method of claim 17, also comprising the following step:
before the step of performing conditional judgment on an extremal ratio of the RR intervals in the ECG signal through a first model to obtain a first score, removing a RR interval that is greater than 0.5 times the mean value and less than 1.6 times the mean value within the preset time.

19. An atrial fibrillation detection system, comprising a memory and one or more processors, wherein the memory is connected with the one or more processors, and instructions executable for the one or more processors are stored in the memory; the instructions are executed by the one or more processors to make the one or more processors execute the method of claim 11.
